(19) United States
(12) Patent Application Publication
(10) Pub. No.: US 2024/0174966 A1
(43) Pub. Date: May 30, 2024

(54) FLUID SAMPLING SYSTEM FOR BIOTECHNOLOGICAL APPLICATIONS, OPERATING METHOD AND USE THEREOF

(71) Applicant: SECURECELL AG, Urdorf (CH)

(72) Inventor: Carlo ANDRETTA

(73) Assignee: SECURECELL AG, Urdorf (CH)

(21) Appl. No.: 18/277,298

(22) PCT Filed: Feb. 25, 2022
(86) PCT No.: PCT/EP2022/054899

§ 371 Date: Aug. 15, 2023

(30) Foreign Application Priority Data
Feb. 25, 2021 (EP) 21159429.6

(51) Int. Cl. C12M 1/26; C12M 1/00; C12M 1/12; C12M 1/34; C12M 3/00

(52) U.S. Cl. CPC C12M 33/14; C12M 23/38; C12M 23/48; C12M 29/10; C12M 29/14; C12M 29/20; C12M 37/04; C12M 41/32

(57) ABSTRACT

A fluid sampling system for biotechnological applications, comprises a bioreactor chamber (2) for a fluid culture medium (4) containing cells (6) and further comprising transfer means (8) for transferring controlled amounts of culture medium from said bioreactor chamber to a target container (10). The transfer means comprise: a perfusion probe (12) with a fluid-tight probe housing surrounding an internal probe volume (14) and having an inlet probe aperture (16) and an outlet probe aperture (18); a fluid filtering element (20) sealingly connected to the probe housing and forming a cover of the inlet probe aperture. A fluid connection line comprises a fluid receptacle (30) disposed between the outlet probe aperture (18) of the perfusion probe and the target container (10) and disposed on a weight measuring station (32) configured for acquisition of a weight signal corresponding to the fluid receptacle's momentary weight.

### FIELD OF THE INVENTION

The present invention generally relates to a fluid sampling system suitable for biotechnological applications. Moreover, the invention relates to a method of operating such a sampling system and to a use thereof.

### BACKGROUND OF THE INVENTION

Fluid sampling systems of various types are widely used in biotechnology. One type of such systems generally comprises a fluid sampling probe immersed in a fluid of interest and means for transferring portions of said fluid to an appropriate fluid receiving section. In order to avoid undesirable sampling of particulate matter such as culture cells but also various types of debris, such sampling probes generally comprise some kind of microfiltration device. Depending on the application, the fluid sampling system is configured for optimum performance with regard to selected aspects such as fluid throughput, accuracy of transferred fluid portions or size range of particles contained in the transferred fluid portions.

In many cases, it would be desirable to have a fluid sampling system which is equipped with a continuously available means for determining the amount of transferred fluid.

### SUMMARY OF THE INVENTION

It is an object of the present invention to provide an improved fluid sampling system suitable for biotechnological applications. In particular, such a device shall overcome the limitations and disadvantages of presently known devices.

According to one aspect of the invention, a fluid sampling system for biotechnological applications comprises a bioreactor chamber for a fluid culture medium containing cells and further comprising transfer means for transferring controlled amounts of culture medium from said bioreactor chamber to a target container. The transfer means comprise: a perfusion probe with a fluid-tight probe housing surrounding an internal probe volume and having an inlet probe aperture and an outlet probe aperture; a fluid filtering element sealingly connected to the probe housing and forming a cover of the inlet probe aperture, the fluid filtering element being formed as at least one monolithic platelet with a primary face and a secondary face opposed thereto, the primary face being in contact with the culture medium when the perfusion probe is inserted into the bioreactor chamber or connected to the bioreactor chamber, and the secondary face being in contact with the internal volume of the perfusion probe, the fluid filtering element comprising an array of microchannels defining a filtering passage between the primary face and the secondary face, the microchannels each having a predetermined opening selected in the range of 0.2 to 64 μm; and fluid driver means for driving culture medium from the bioreactor chamber through the perfusion probe to yield filtered culture medium, and for driving said filtered culture medium through a fluid connection line into said target container. The fluid connection line comprises a fluid receptacle disposed between the outlet probe aperture of the perfusion probe and the target container. The fluid receptacle is disposed on a weight measuring station configured for acquisition of a weight signal corresponding to the fluid receptacle's momentary weight.

According to a further aspect, there is provided a method of operating the fluid sampling system as defined above, the method comprising the following steps:
a) loading the bioreactor chamber with a fluid culture medium containing cells;
b) after a predetermined time, transferring an amount of filtered culture medium into the fluid receptacle and acquiring a weight signal corresponding to the fluid receptacle's momentary weight,
c) further transferring said amount of filtered culture medium to the target container.

According to another aspect, a fluid sampling system as defined above is used for directing filtered culture medium to one of the following:
a glucose determination station;
a multiple sample collection station;
a harvesting station.

The term “fluid sampling” is not limited to withdrawal of small fluid volumes typically used when taking a sample, i.e., an amount of fluid for analysis. It shall also include comparatively larger fluid volumes useful for production purposes.

The invention is not limited to embodiments with the primary face being in direct contact with the culture medium when the perfusion probe is inserted into the bioreactor chamber. It also includes embodiments wherein the primary face is in contact with the culture medium when it is connected to the bioreactor chamber by appropriate connection means, particularly with a suitable additional fluidic container.

The fluid filtering element is formed as at least one monolithic platelet with an external face and an internal face opposed thereto, the internal face being in contact with the internal volume of the perfusion chamber. The fluid filtering element comprises an array of microchannels defining a filtering fluid passage between the external face and the internal face. The microchannels have a predetermined opening selected in the range of 0.2 to 64 μm. It is highly preferable that all of the microchannels have exactly the same opening within production tolerance, which means a variation of 10% or even substantially less.

The optimum size of the microchannels will depend on the particular application. In general, it will be selected in the range of 0.2 to 10 μm. The lower limit is primarily determined by the available forming technology, but also by the need to have sufficient throughput. The upper limit is determined by the size of particles that should be prevented from entering into the microchannels. For certain applications where it is necessary to prevent bacteria from passing the microfiltration device, the microchannels should have an opening not exceeding about 0.45 μm. For other applications, the microchannels preferably have an opening in the range of 0.9 to 2.2 μm, particularly around 1.6 μm. Openings in the range of 0.2 to 32 μm, particularly in the range of 4 to 32 μm are typically used for cell filtration, i.e., to extract fluid from a bioreactor chamber.

The term “opening” shall be understood as the diameter in the case of microchannels with circular cross section; for non-circular microchannels the term “opening” shall be understood as the smallest transversal size of the cross section. Currently available technologies for forming openings with the above-mentioned diameter range usually require a height to diameter ratio (“aspect ratio”) of up to 5. In other words, the thickness of the front platelet in the region surrounding the microchannels needs to be small enough, i.e., in the range of 1 to 50 μm depending on the microchannel diameter. In order to provide sufficient stiffness of the front platelet, in certain embodiments there are provided reinforcing regions with a substantially higher thickness at locations displaced from the microchannels.

The devices of the present invention are generally intended for biotechnological applications, which implies compatibility with aqueous media and also suitability for sterilization and for cleaning operations. Accordingly, the devices are advantageously made of appropriate materials such as e.g., stainless steel of appropriate grade. Moreover, the terms “fluid-safe manner” and “sealingly connected” shall imply technical solutions which can prevent unwanted fluid transfer at the temperature and pressure conditions prevailing in a biotechnological setup, such as in a bioreactor. This is generally achievable with parts made of stainless steel, silicon and possibly glass, and with O-ring seals using appropriate elastomeric materials. Metallic seals are also usable.

An important component of the present invention is the inclusion of a weight measuring station, which allows continuous monitoring of amounts of fluid being transferred in the system.

Advantageous embodiments are defined in the dependent claims and are described below.

In principle, various types of weight measuring station could be used. According to a particularly advantageous embodiment (claim 2), the weight measuring station comprises a bending beam load cell. Such devices are commercially available in various configurations and for various weight ranges.

Advantageously (claim 3), the weight measuring station comprises a rigid beam which has a first beam end and a second beam end and which is pivotably attached to a holder base of the weight measuring station at a pivoting point located between said first and second end, wherein the fluid receptacle rests on the first beam end thereby exerts a downward force Fd, and wherein the second beam end transmits a corresponding upward force Fu to the load cell. In this manner, the use of highly compact and conveniently available bending beam load cells is possible while still providing an appropriate accuracy of the weight measurement.

According to one embodiment (claim 4), the fluid receptacle comprises: a fluid-tight receptacle chamber provided with a fluid entrance connector located in an upper region of the receptacle chamber, a fluid exit connector located in a bottom region of the receptacle chamber, and a gas pressure compensation port located in an upper region of the receptacle chamber. It is particularly advantageous if the bottom region has a tapering cross section with an inner diameter progressively decreasing in downwards direction. In certain embodiments, the fluid connectors are of the so-called Luer type.

To enable the necessary basic fluid handling operations, the fluid driver means advantageously comprise (claim 5): a bidirectional fluid pump disposed between the outlet probe aperture and the fluid entrance connector, and a monodirectional fluid pump disposed between the fluid exit connector and the target container. According to an advantageous embodiment (claim 6), the fluid driver means further comprise: a first fluid switch disposed between the outlet probe aperture and said bidirectional fluid pump for selectively connecting a washing fluid reservoir to said bidirectional pump; and a second fluid switch disposed between said said monodirectional fluid pump and the target container for selectively connecting the monodirectional fluid pump to a waste container.

According to a particularly advantageous embodiment (claim 7), each fluid filtering element comprises a frame region with a first thickness D1, and the microchannel array is disposed in at least one core region surrounded by the frame region, the core region having a second thickness D2 which is substantially smaller than the first thickness. For example, the frame region may have a first thickness D1 of the order of 0.3 to 0.8 mm, particularly about 0.5 mm, whereas the core region may have a second thickness D2 of merely 0.005 to 0.010, particularly about 0.008 mm.

Furthermore, it is advantageous (claim 8) if the microchannel array comprises a plurality of array segments, with neighboring segments being separated by a separation rib having a third thickness D3 which is substantially equal to the first thickness D1.

Advantageously, the fluid filtering element is made of a material that is suitable for photolithographic processing, such as silicon (Si) or silicon nitride (Si₃N₄), which is a very convenient technique for forming narrow structures with a well-defined shape. In one embodiment (claim 9), each fluid filtering element is made of silicon and is sealingly connected to the probe housing by gluing or by welding. In some embodiments, the fluid transmission element is functionalized, i.e., it is provided with a suitable coating. The type and thickness of such coating will depend on the particular application.

According to a particularly advantageous embodiment (claim 10), the probe housing is substantially square-tubed and comprises a pair of mutually parallel first lateral faces and a pair of mutually parallel second lateral faces, the lateral faces of each pair being perpendicular to the lateral faces of the other pair, wherein the first lateral faces are configured to form said inlet probe aperture, and wherein the second lateral faces are configured to form said outlet probe aperture. According to a further embodiment (claim 11), the probe housing comprises a plurality of at least two separate probe compartments disposed along a tubular axis L, wherein each compartment comprises a respective pair of first lateral faces and second lateral faces. According to yet another embodiment (claim 12), the second lateral faces are provided with longitudinal grooves, each groove being connected to the respective probe compartment and to a respective fluid outlet port, each second lateral face being covered by a lateral covering pad.

According to an advantageous embodiment (claim 14), the method of operating a specific embodiment of the fluid sampling system further comprises the steps of: selectively connecting the washing fluid reservoir to said bidirectional pump; and selectively connecting said monodirectional fluid pump to the waste container, followed by driving of washing fluid through the fluid receptacle; the above steps being carried out at least before the sequence of steps a) to c).

It is possible to manage the fluid withdrawal but also fluid supply, e.g., in order to periodically backflush the fluid filtering element, by means of a single fluid connection line. As will be understood, such a connection line is managed by an appropriate fluid handling system. Such a system is typically configured to be able to perform at least the following steps:
a) withdrawing filtered fluid from the internal chamber volume;
b) running backflushing medium through the buffer volume.

Indeed, it has turned out that a protocol relying on a stepwise operation with 10 time steps withdrawal followed by 1 time step backflush allows for a highly efficient prevention of clogging by debris cake formation on the surface of the fluid filtering element.

As an additional measure to prevent clogging, one can apply some continuous stirring to cause a substantial parallel medium flow over the surface of the fluid transmission elements.

### BRIEF DESCRIPTION OF THE DRAWINGS

The above mentioned and other features and objects of this invention and the manner of achieving them will become more apparent and this invention itself will be better understood by reference to the following description of various embodiments of this invention taken in conjunction with the accompanying drawings, wherein:

FIG. 1 shows one embodiment of a fluid sampling system for biotechnological applications, in a schematic view;

FIG. 2 shows a weight measuring station of a fluid sampling system, including a fluid receptacle, in a schematic vertical sectional view;

FIG. 3 shows a fluid receptacle, in a partially disassembled state, in a perspective view;

FIG. 4 shows the fluid receptacle of FIG. 3, in an assembled state, in a vertical sectional view;

FIG. 5 a weight measuring station of a fluid sampling system, in a partially disassembled state, in a perspective view;

FIG. 6 the weight measuring station of FIG. 5, in an assembled state, in a perspective view;

FIG. 7 a mounting unit comprising a weight measuring station and a fluid receptacle, in a perspective view;

FIG. 8 shows a perfusion probe, in a perspective view;

FIG. 9 shows a fluid transmission element of the perfusion probe of FIG. 8, in a top view;

FIG. 10 shows the the fluid transmission element of FIG. 9 in a sectional view according to section A-A of FIG. 9; and

FIG. 11 shows an enlarged portion B of FIG. 10;

FIG. 12 shows the perfusion probe of FIG. 8, in a disassembled state, in a perspective view;

FIG. 13 shows a tip portion of the perfusion probe of FIG. 8, in a top view;

FIG. 14 shows the tip portion of FIG. 13, in a sectional view according to section A-A of FIG. 13;

FIG. 15 shows the tip portion of FIG. 13, with lateral covering pad removed, in a first side elevational view;

FIG. 16 shows the tip portion of FIG. 13, in a sectional view according to section B-B of FIG. 13;

FIG. 17 shows the tip portion of FIG. 13, with lateral covering pad removed, in a second side elevational view;

FIG. 18 shows the tip portion of FIG. 13, in a sectional view according to section C-C of FIG. 13; and

FIG. 19 shows another embodiment of a fluid sampling system for biotechnological applications, in a schematic view.

### DETAILED DESCRIPTION OF THE INVENTION

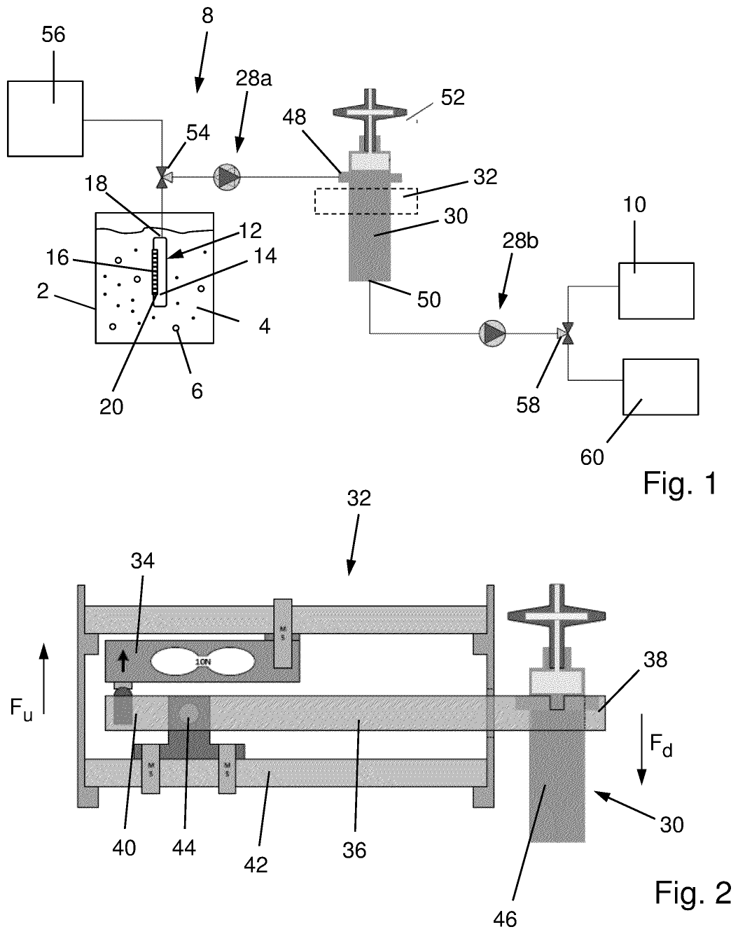

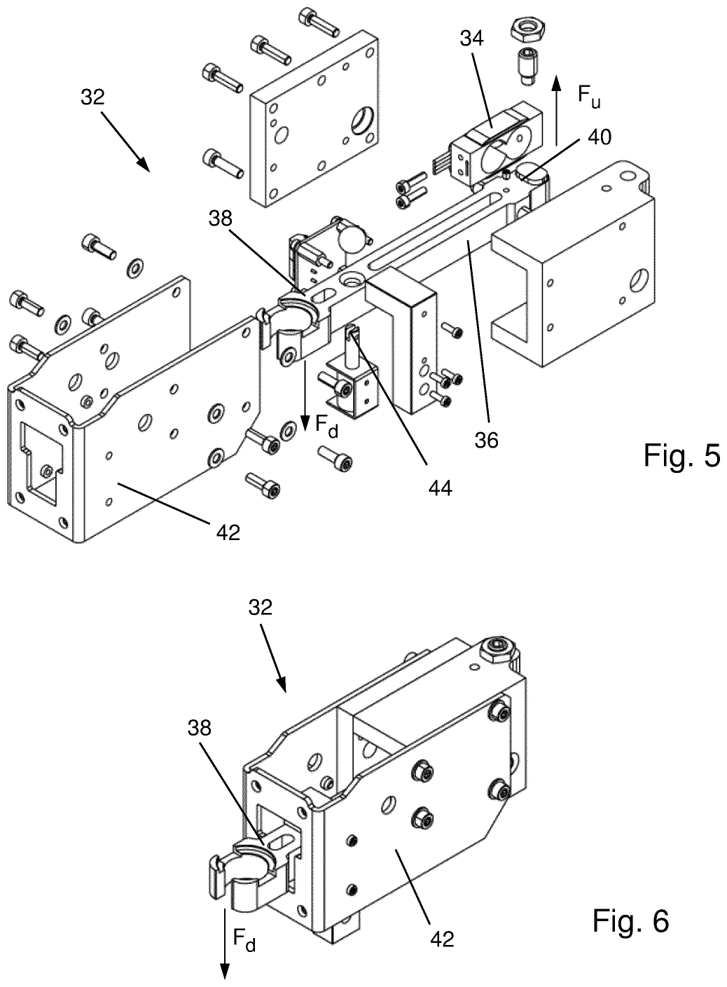

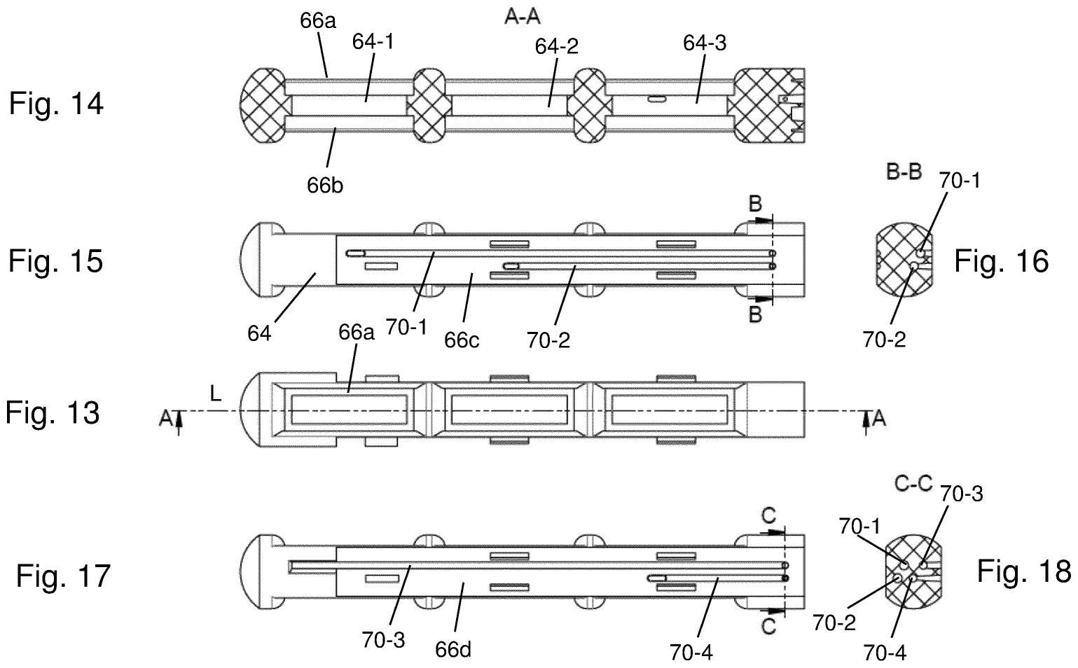

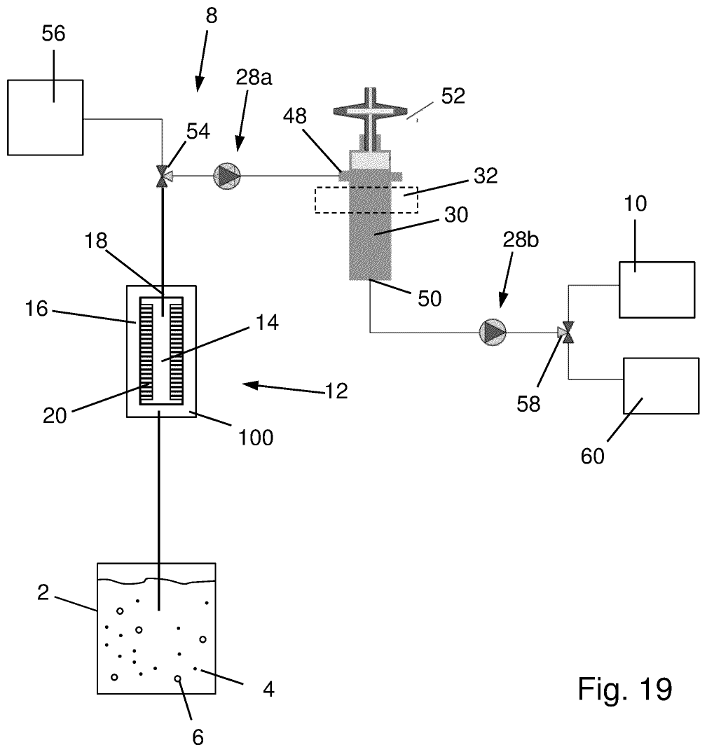

In order to better explain the general principle of the present invention, FIG. 1 shows an overall schematic view of a fluid sampling system. Moreover, FIG. 2 shows a schematic view of a weight measuring station of a fluid sampling system, including a fluid receptacle. Further details of the invention are shown in FIGS. 3 to 18 and in FIG. 19.

As shown in FIGS. 1 and 2, a fluid sampling system for biotechnological applications, comprises a bioreactor chamber (2) for a fluid culture medium (4) containing cells (6) and further comprises transfer means (8) for transferring controlled amounts of culture medium from the bioreactor chamber to a target container (10). The transfer means comprise:
a perfusion probe (12) with a fluid-tight probe housing surrounding an internal probe volume (14) and having an inlet probe aperture (16) and an outlet probe aperture (18);
a fluid filtering element (20) sealingly connected to the probe housing and forming a cover of the inlet probe aperture, the fluid filtering element being formed as at least one monolithic platelet with a primary face (22) and a secondary face (24) opposed thereto, the primary face being in contact with the culture medium when the perfusion probe is inserted into the bioreactor chamber or connected to the bioreactor chamber, and the secondary face being in contact with the internal volume of the perfusion probe, the fluid filtering element comprising an array of microchannels (26) defining a filtering passage between the primary face and the secondary face, the microchannels each having a predetermined opening selected in the range of 0.2 to 64 μm;
fluid driver means (28a, 28b) for driving culture medium from the bioreactor chamber through the perfusion probe to yield filtered culture medium, and for driving said filtered culture medium through a fluid connection line into said target container.

The fluid connection line comprises a fluid receptacle (30) disposed between the outlet probe aperture (18) of the perfusion probe and the target container (10), the fluid receptacle (30) being disposed on a weight measuring station (32) configured for acquisition of a weight signal corresponding to the fluid receptacle's momentary weight.

In the embodiment shown in FIGS. 2, 5 and 6, the weight measuring station comprises a bending beam load cell (34). The weight measuring station (32) further comprises a rigid beam (36) which has a first beam end (38) and a second beam end (40) and which is pivotably attached to a holder base (42) of the weight measuring station at a pivoting point (44) located between said first and second end, wherein the fluid receptacle rests on the first beam end thereby exerts a downward force (Fd), and wherein the second beam end transmits a corresponding upward force (Fu) to the load cell (34).

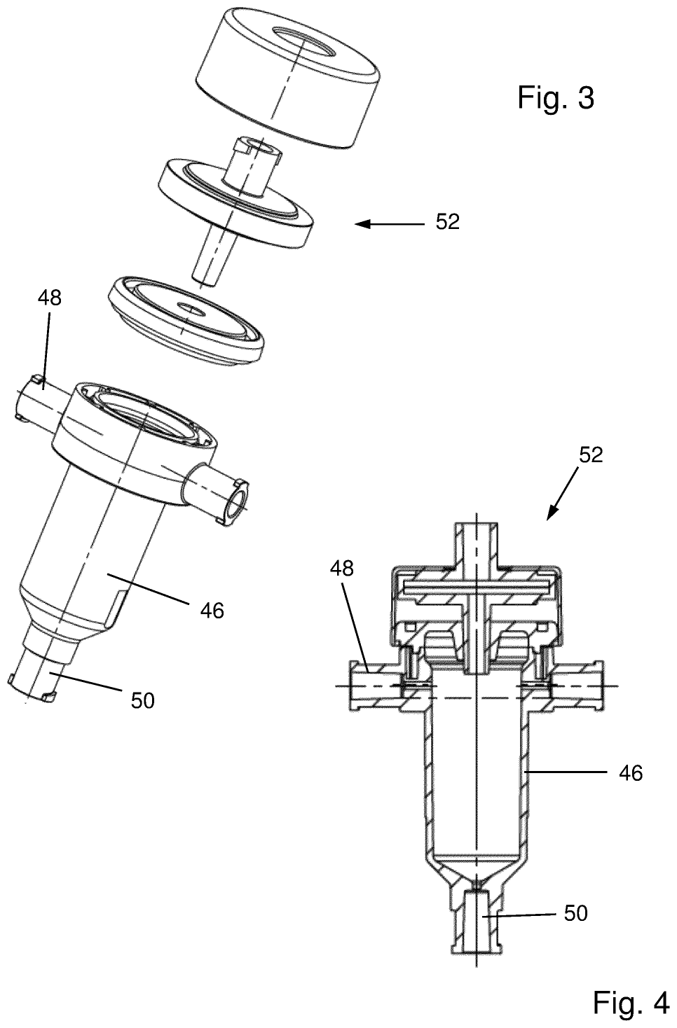

As shown in FIGS. 3 and 4, the fluid receptacle (30) comprises a fluid-tight receptacle chamber (46) provided with a fluid entrance connector (48) located in an upper region of the receptacle chamber, a fluid exit connector (50) located in a bottom region of the receptacle chamber, and a gas pressure compensation port (52) located in an upper region of the receptacle chamber.

Reverting to FIG. 1 again, the fluid driver means further comprise a first fluid switch (54) disposed between the outlet probe aperture (18) and the bidirectional fluid pump (28a) for selectively connecting a washing fluid reservoir (56) to the bidirectional pump (28a), and a second fluid switch (58) disposed between said said monodirectional fluid pump (28b) and the target container (10) for selectively connecting the monodirectional fluid pump (28b) to a waste container (60).

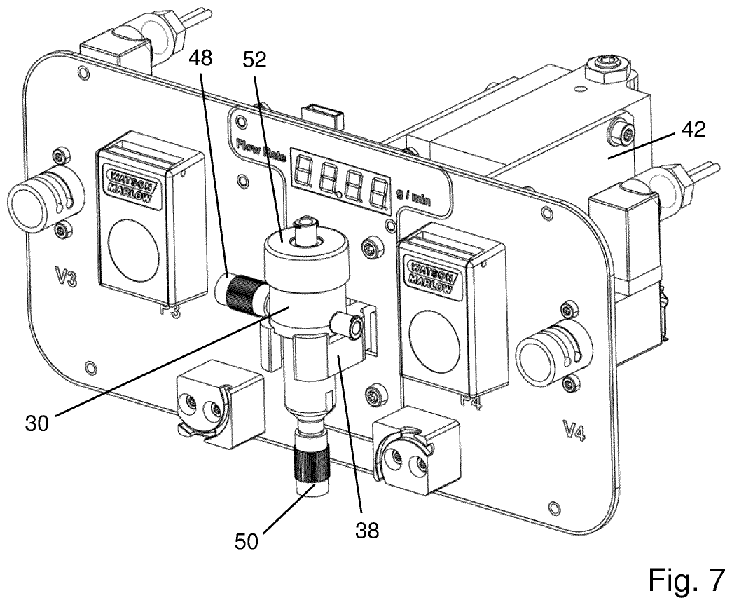

To further illustrate the invention, FIG. 7 shows a mounting unit comprising a weight measuring station and a fluid receptacle.

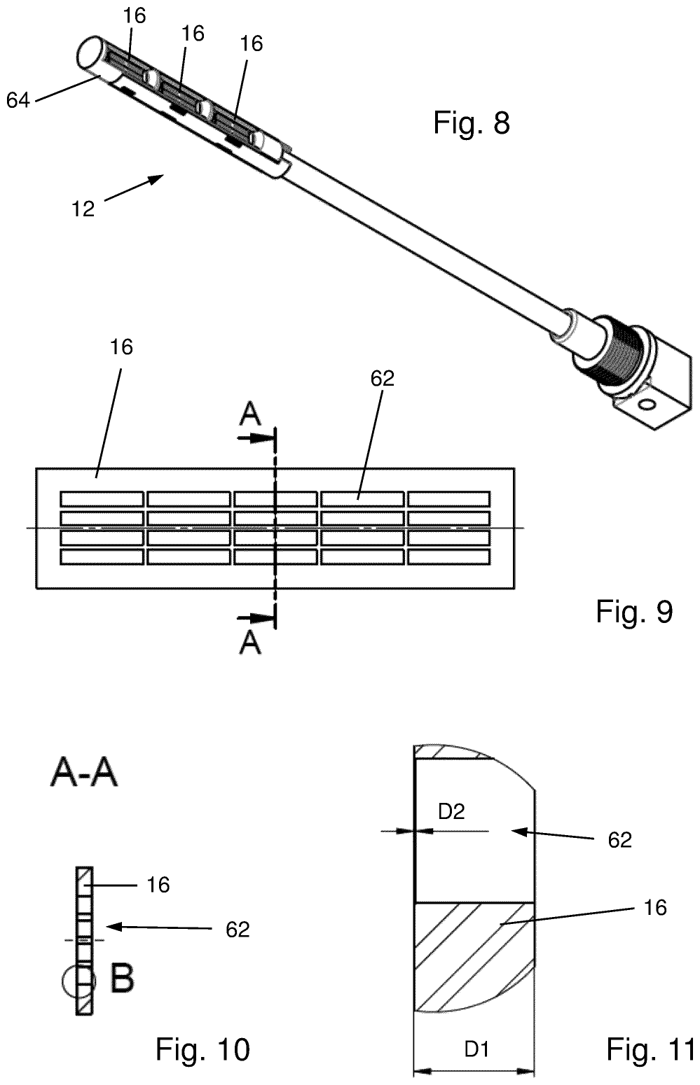

Further details of a perfusion probe are shown in FIGS. 8 to 18.

Each fluid filtering element (16) comprises a frame region with a first thickness (D1) and wherein the microchannel array is disposed in at least one core region (62) surrounded by the frame region, the core region having a second thickness (D2) which is substantially smaller than the first thickness. The microchannel array comprises a plurality of array segments, with neighboring segments being separated by a separation rib having a third thickness (D3) which is substantially equal to the first thickness (D1).

In the embodiment shown, each fluid filtering element is made of silicon (Si) and is sealingly connected to the probe housing (64) by gluing or by welding.

The probe housing (64) is substantially square-tubed and comprises a pair of mutually parallel first lateral faces (66a, 66b) and a pair of mutually parallel second lateral faces (66c, 66d), the lateral faces of each pair being perpendicular to the lateral faces of the other pair, wherein the first lateral faces (66a, 66b) are configured to form said inlet probe aperture (16), and wherein the second lateral faces (66c, 66d) are configured to form said outlet probe aperture (18).

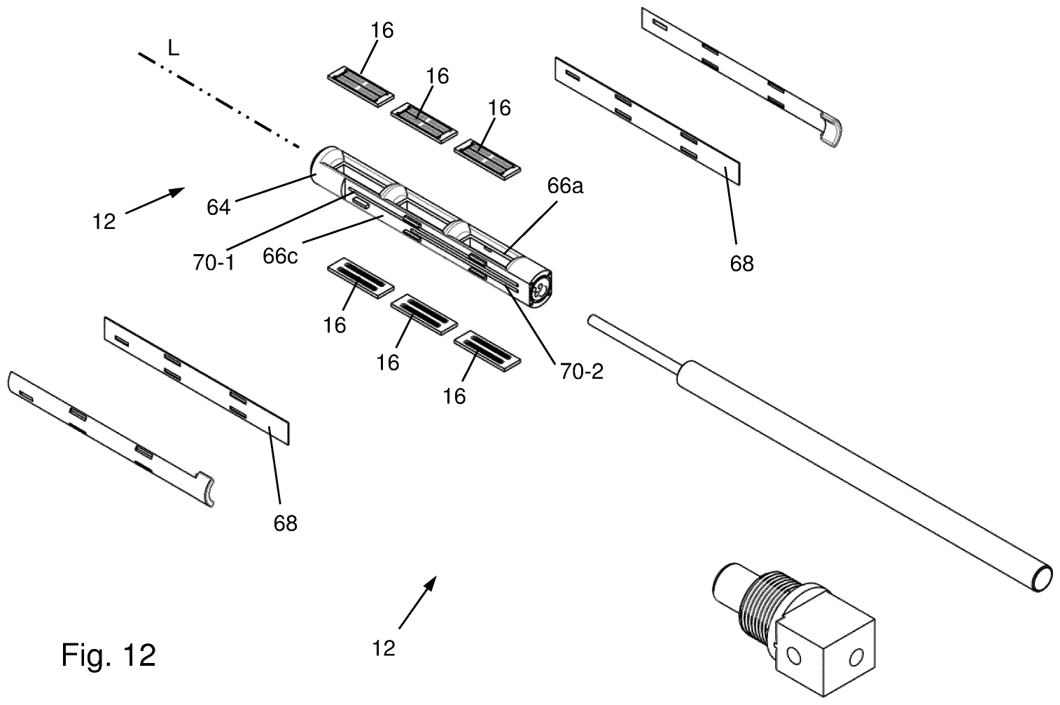

In the embodiment shown in FIG. 12, the probe housing (64) comprises a plurality of at least two separate probe compartments (64-1, 64-2, 64-3) disposed along a tubular axis L, wherein each compartment comprises a respective pair of first lateral faces and second lateral faces. In particular, the second lateral faces (66c, 66d) are provided with longitudinal grooves (70-1, 70-2, 70-3, 70-4), each groove being connected to a respective probe compartment (64-1, 64-2, 64-3) and to a respective fluid outlet port (72), each second lateral face being covered by a lateral covering pad (68).

When operating the system described above, the following steps are executed:
a) loading the bioreactor chamber (2) with a fluid culture medium (4) containing cells (6);
b) after a predetermined time, transferring an amount of filtered culture medium into the fluid receptacle (30) and acquiring a weight signal corresponding to the fluid receptacle's momentary weight,
c) further transferring said amount of filtered culture medium to the target container (10).

In one embodiment, the mode of operation further comprises the steps of:
selectively connecting the washing fluid reservoir (56) to said bidirectional pump (28a);
and selectively connecting said monodirectional fluid pump (28b) to the waste container (60), followed by driving of washing fluid through the fluid receptacle (30);
noting that the above steps shall be carried out at least before the sequence of steps a) to c).

A further embodiment of a fluid sampling system for biotechnological applications is shown in FIG. 19. In contrast to the system shown in FIG. 1, the perfusion probe (12) is not directly immersed into the bioreactor (2). Instead, the perfusion probe is immersed into a media container (100) which is in fluid communication with the bioreactor (2) by means of a connection tube, particularly a flexible tube. Moreover, in the example of FIG. 19, the perfusion probe (12) comprises two fluid filtering elements (20). This embodiment allows substantially increased fluid throughput as compared to the embodiment of FIG. 1 because the effective area of the fluid filtering elements (20) can be scaled up.

1. A fluid sampling system for biotechnological applications, comprising a bioreactor chamber for a fluid culture medium containing
cells and further comprising
transfer means for transferring controlled amounts of the fluid culture medium from said bioreactor chamber to a target container, wherein
the transfer means comprise(s)
a perfusion probe with a fluid-tight probe housing surrounding an internal probe volume and having an inlet probe aperture and an outlet probe aperture;
a fluid filtering element sealingly connected to the fluid-tight probe housing and forming a cover of the inlet probe aperture, the fluid filtering element being formed as at least one monolithic platelet with a primary face and a secondary face opposed thereto,
the primary face being in contact with the fluid culture medium when the perfusion probe is inserted into the bioreactor chamber or connected to the bioreactor chamber, and
the secondary face being in contact with the internal probe volume of the perfusion probe, the fluid filtering element comprising an array of microchannels defining a filtering passage between the primary face and the secondary face, the microchannels each having a predetermined opening selected in a range of 0.2 to 64 μm;
fluid driver means for driving the fluid culture medium from the bioreactor chamber through the perfusion probe to yield filtered culture medium, and for driving said filtered culture medium through a fluid connection line into said target container;
wherein the fluid connection line comprises a fluid receptacle disposed between the outlet probe aperture of the perfusion probe and the target container,
wherein
the fluid receptacle is disposed on a weight measuring station configured for acquisition of a weight signal corresponding to a momentary weight of the fluid receptacle.

2. The fluid sampling system according to claim 1, wherein the weight measuring station comprises a bending beam load cell.

3. The fluid sampling system according to claim 2, wherein the weight measuring station comprises a rigid beam which has a first beam end and a second beam end and which is pivotably attached to a holder base of the weight measuring station at a pivoting point located between said first beam end and second beam end, wherein the fluid receptacle rests on the first beam end and thereby is configured to excert a downward force (Fd), and wherein the second beam end is configured to transmit a corresponding upward force (Fu) to the load cell.

4. The fluid sampling system according to claim 1, wherein the fluid receptacle comprises
a fluid-tight receptacle chamber provided with a fluid entrance connector located in an upper region of the receptacle chamber,
a fluid exit connector located in a bottom region of the receptacle chamber, and
a gas pressure compensation port located in the upper region of the receptacle chamber.

5. The fluid sampling system according to claim 4, wherein said fluid driver means comprise(s):
a bidirectional fluid pump disposed between the outlet probe aperture and the fluid entrance connector, and
a monodirectional fluid pump disposed between the fluid exit connector and the target container.

6. The fluid sampling system according to claim 5, wherein said fluid driver means further comprise(s):
a first fluid switch disposed between the outlet probe aperture and said bidirectional fluid pump for selectively connecting a washing fluid reservoir to said bidirectional pump; and
a second fluid switch disposed between said monodirectional fluid pump and the target container for selectively connecting the monodirectional fluid pump to a waste container.

7. The fluid sampling system according to claim 1, wherein each fluid filtering element comprises a frame region with a first thickness and wherein the microchannel array is disposed in at least one core region surrounded by the frame region, the core region having a second thickness which is substantially smaller than the first thickness.

8. The fluid sampling system according to claim 7, wherein the microchannel array comprises a plurality of array segments, with neighboring segments being separated by a separation rib having a third thickness which is substantially equal to the first thickness.

9. The fluid sampling system according to claim 7, wherein each fluid filtering element is made of silicon (Si), and is sealingly connected to the fluid-tight probe housing by gluing or by welding.

10. The fluid sampling system according to claim 9, wherein the fluid-tight probe housing is substantially square-tubed and comprises a pair of mutually parallel first lateral faces and a pair of mutually parallel second lateral faces, the lateral faces of each pair being perpendicular to the lateral faces of the other pair, wherein the first lateral faces are configured to form said inlet probe aperture, and wherein the second lateral faces are configured to form said outlet probe aperture.

11. The fluid sampling system according to claim 10, wherein the fluid-tight probe housing comprises a plurality of at least two separate probe compartments disposed along a tubular axis, wherein each compartment comprises a respective pair of first lateral faces and second lateral faces.

12. The fluid sampling system according to claim 10, wherein the second lateral faces are provided with longitudinal grooves, each groove being connected to a respective probe compartment and to a respective fluid outlet port, each second lateral face being covered by a lateral covering pad.

13. A method of operating the fluid sampling system according to claim 1, comprising:
a) loading the bioreactor chamber with the fluid culture medium containing the cells;
b) after a predetermined time, transferring an amount of the filtered culture medium into the fluid receptacle and acquiring a weight signal corresponding to a momentary weight of the fluid receptacle,
c) further transferring said amount of the filtered culture medium to the target container.

14. A method of operating the fluid sampling system according to claim 6, comprising:
a) loading the bioreactor chamber with the fluid culture medium containing the cells;
b) after a predetermined time, transferring an amount of the filtered culture medium into the fluid receptacle and acquiring a weight signal corresponding to a momentary weight of the fluid receptacle,
c) further transferring said amount of the filtered culture medium to the target container,
d) selectively connecting the washing fluid reservoir to said bidirectional pump; and selectively connecting said monodirectional fluid pump to the waste container, followed by driving of washing fluid through the fluid receptacle; the above steps being carried out at least before a) to c), with a) to c) being carried out in sequence.

15. A method comprising
providing the fluid sampling system according to claim 1 which produces the filtered culture medium from the fluid culture medium containing the cells and directing the filtered culture medium to one of the following:
a glucose determination station;
a multiple sample collection station; or
a harvesting station.

16. The fluid sampling system according to claim 2, wherein the fluid receptacle comprises
a fluid-tight receptacle chamber provided with a fluid entrance connector located in an upper region of the receptacle chamber,
a fluid exit connector located in a bottom region of the receptacle chamber, and
a gas pressure compensation port located in an upper region of the receptacle chamber.

17. The fluid sampling system according to claim 3, wherein the fluid receptacle comprises
a fluid-tight receptacle chamber provided with a fluid entrance connector located in an upper region of the receptacle chamber,
a fluid exit connector located in a bottom region of the receptacle chamber, and
a gas pressure compensation port located in an upper region of the receptacle chamber.

18. The fluid sampling system according to claim 6, wherein each fluid filtering element comprises a frame region with a first thickness and wherein the microchannel array is disposed in at least one core region surrounded by the frame region, the core region having a second thickness which is substantially smaller than the first thickness.

19. The fluid sampling system according to claim 18, wherein the microchannel array comprises a plurality of array segments, with neighboring segments being separated by a separation rib having a third thickness which is substantially equal to the first thickness.

20. The fluid sampling system according to claim 8, wherein each fluid filtering element is made of silicon (Si), and is sealingly connected to the fluid-tight probe housing by gluing or by welding.
